(19) United States
(12) Patent Application Publication
(10) Pub. No.: US 2007/0003584 A1
(43) Pub. Date: Jan. 4, 2007

(54) Liquid crystal polymer syringes and containers and methods of use for long term storage of filler materials

(76) Inventor: Russell Anderson, San Diego, CA (US)

Correspondence Address: HELLER EHRMAN LLP, 4350 LA JOLLA VILLAGE DRIVE #700, 7TH FLOOR, SAN DIEGO, CA, 92122, US

(21) Appl. No.: 11/454,537

(22) Filed: Jun. 16, 2006

Related U.S. Application Data
(60) Provisional application No. 60/691,506, filed on Jun. 16, 2005.

(51) Int. Cl. A61K 8/02; A61M 31/00

(52) U.S. Cl. 424/401; 604/500

(57) ABSTRACT

A syringe or container including a barrier surface for the long term storage of filler compositions and pre-filled syringes having a barrier surface that include filler compositions are provided.

### REFERENCE TO PRIORITY DOCUMENT

This application claims the benefit of priority to U.S. Provisional Patent Application Ser. No. 60/691,506 entitled “Liquid Crystal Polymer Syringe”, filed Jun. 16, 2005, herein incorporated by reference.

### BACKGROUND OF THE INVENTION

1. Field of the Invention

The present invention relates generally to syringes and other containers comprising liquid crystal polymer and methods for the long term storage of materials in said syringes and other containers.

2. Background

Fillers, such as soft tissue fillers, have a variety of applications for tissue augmentation and tissue bulking. Physiological conditions of organisms (e.g., mammals) can be treated by way of insertions (e.g., injections) of fillers into tissues using insertion devices such as syringes. Typical instances include treating a condition by injecting a filler material into and/or adjacent to tissue treatment sites.

Tissue treatment sites can be, for example, sphincters (e.g., urinary sphincter muscles at bladder necks or lower esophageal sphincter muscles) or epidermal indentations (e.g., wrinkles) or other irregularities or undesired features, and can be inserted (e.g., implanted) to, for example, bulk-up, even-out, or otherwise affect or treat an appearance or condition of tissue. For instance, an appearance of wrinkles can be attenuated, or a functionality of a sphincter can be augmented.

Treating a loss of bladder control, which is commonly associated with, for example, stress urinary incontinence, can include injecting filler material into and/or adjacent to a urinary sphincter muscle at the bladder neck to thereby provide a bulking effect to the treated tissue and assist in closure of the urinary sphincter. Another treatable condition is acid reflux, which is commonly recognized as is a digestive disorder in which the lower esophageal sphincter connecting the esophagus to the stomach malfunctions and allows stomach contents to flow up through the lower esophageal sphincter into the esophagus. Treatment of acid reflux can be accomplished by way of injecting a filler material into the lower esophageal sphincter to reduce or eliminate the undesirable passage of stomach contents into the esophagus. Additionally, treating cosmetic defects in the skin can comprise injecting filler materials into various layers of the skin of the patient as dermal fillers. The dermal fillers can be injected with a device such as a syringe through an attached needle or through a lumen with a needle attached to the end of the lumen.

ArteColl® and ArteFill® are trade names for tissue bulking or filling agents, such as collagen-suspended microspheres, which can be formed of polymers such as polymethyl methacrylate (PMMA) and which can be implanted via, for example, injections. Examples of such microsphere-based filler materials are disclosed in U.S. Pat. No. 5,344,452, which issued on Sep. 6, 1994 and the entire contents of which is incorporated herein by reference.

If the filler solution or mixture (e.g., suspension) is stored in conventional containers or syringes that are formed from, for example, polypropylene, the solution or mixture may leach or escape through walls of the container or syringe. For example, bovine collagen and PMMA microspheres held in a syringe made of polypropylene leaches approximately 20% of the water in the collagen solution over the course of a year, That leaching results in not only a loss of contents, but an uncontrollable increase in the concentration of constituents in solution (ie; collagen or lidocaine hydrochloride).

Glass syringes or containers can reduce leaching of a solution or mixture (e.g., suspension). However, potentially toxic heavy metals can leach out of the glass and can contaminate the solution or mixture that is contained in the glass syringe or container. When the solution or suspension containing the heavy metals is implanted (e.g., injected) into, for example, the skin or sphincter area of the patient, the health of the patient and/or a success of a procedure may be compromised or adversely (e.g., undesirably or unpredictably) affected by the presence of such heavy metals.

Thus, there is a need for syringes and containers that do not leach contaminants or absorb filler compositions and offer a stable, long-term storage environment. Furthermore, there is a need for methods for convenient long-term storage of filler composition. The present invention satisfies these needs and provides further advantages.

### SUMMARY

In accordance with the invention, a syringe or container made of a barrier material sufficiently resistant to absorption of syringe or container contents is provided. The barrier material can have vapor or moisture barrier characteristics. The barrier material can be essentially free of heavy metals. For a syringe, the barrier material can coat contents-contacting surfaces of a barrel, plunger, needle or interior sleeve or any combination thereof. The barrier material can be, at least in part, a liquid crystal polymer composition. The liquid crystal polymer composition can include, but is not limited to, styrene methylmethacrylate co polymer (such as ZYLAR®), ZENITE® (a proprietary formulation), or cyclic olefin copolymer (COP) of ethylene and norbornene (such as TOPAS®). The barrel, plunger, needle, or interior sleeve or any combination thereof, can be, at least in part, a liquid crystal polymer composition.

Also provided is a pre-filled syringe or container made of a barrier material sufficiently resistant to leaching or absorption or both. The filler can be polymethylmethacrylate (PMMA) microspheres. The PMMA microspheres can be formulated with collagen. Such formulations can be ArteFill®.

Also provided are methods for storing filler material by filling a syringe or container made of, at least in part, a liquid crystal polymer with filler material and sealing the syringe to prevent escape of filler material. The sealing step can include using syringe components that have liquid crystal polymer compositions coating the filler material contacting surfaces, such as the barrel, the plunger, or the needle, individually or in combination. The filler can be polymethylmethacrylate (PMMA) microspheres. The PMMA microspheres can be formulated with collagen. Such formulations can be ArteFill®.

Other features and advantages of the present invention should be apparent from the following description of the disclosed embodiment, taken in conjunction with the accompanying drawing, which illustrate, by way of example, the principles of the invention.

### BRIEF DESCRIPTION OF THE DRAWINGS

FIG. 1 shows a syringe.

### DETAILED DESCRIPTION

An aspect of the present invention includes the provision of a syringe or other container for the solution or mixture (e.g., suspension) comprising filler compositions, including soft tissue filler compositions. Soft tissue filler compositions can include, for example, microspheres, such as collagen-suspended microspheres, which can be formed of polymers such as polymethyl methacrylate (PMMA). Examples of such microsphere-based filler materials are disclosed in U.S. Pat. No. 5,344,452, which issued on Sep. 6, 1994 and the entire contents of which is incorporated herein by reference. In one embodiment, the soft tissue filler is ArteFill®, which is approximately 20% by weight PMMA and approximately 80% by weight a composition of 3.5% purified bovine collagen, 2.7% phosphate buffer, 0.9% sodium chloride, 0.3% lidocaine hydrochloride, and 92.6% water for injection. Filler materials can be cross-linked or not cross-linked, or made of a synthetic and/or polymeric material, such as, for example, polylactic acid, organic compounds, inorganic compounds, ceramic materials, polymethacrylate, polypropylene, polytetrafluoroethylene (PTFE), and combinations thereof. Other soft tissue fillers include, but are not limited to, collagen; hollow cylinder pellets as disclosed in U.S. Patent Publication No. 2004/210230, entitled “Materials and Methods for Soft Tissue Augmentation”; polysaccharide-based gel as disclosed in U.S. Patent Publication No. 2004/0047892, entitled “Filler Composition for Soft Tissue Augmentation and Reconstructive Surgery”; polyhydroxyalkanoate materials as disclosed in U.S. Pat. Nos. 6,585,994 and 6,555,123, entitled “Polyhydroxyalkanoate Compositions for Soft Tissue Repair, and Viscosupplementation”; crosslinked hyaluronic acid as disclosed in U.S. Pat. No. 5,827,937; repetitive protein polymers as disclosed in as disclosed in U.S. Patent Publication No. 2003/0176355, entitled “Synthetic Proteins for In Vivo Drug Delivery and Tissue Augmentation”; a three-part injectable polymer as disclosed in U.S. Pat. No. 5,785,642; a two-part injectable polymer as disclosed in U.S. Pat. No. 6,312,725; keratin as disclosed in U.S. Pat. No. 5,712,252; ceramic microsphere compositions as disclosed in U.S. Pat. Nos. 5,922,025 and 6,432,437, and 6,537,574, entitled “Soft Tissue Augmentation Material”; biocompatible tissue-reactive prepolymer as disclosed in U.S. Pat. No. 6,702,731, entitled “Situ Bulking Device”; cross-linked blood plasma proteins as disclosed in U.S. Pat. No. 7,015,198, entitled “Materials for Soft Tissue Augmentation and Methods of Making and Using Same”; radiation cross-linked hydrogels as disclosed in U.S. Pat. No. 6,537,569, entitled “Radiation Cross-Linked Hydrogels”; bioelastomers as disclosed in U.S. Pat. Nos. 6,533,819 and 6,699,294, entitled “Injectable Implants for Tissue Augmentation and Restoration”; cross-linked water-swellable polymer particles as disclosed in U.S. Pat. Nos. 6,214,331 and 6,544,503, entitled “Process for the Preparation of Aqueous Dispersions of Particles of Water-Soluble Polymers and Particles Obtained”; and compositions including a pseudoplastic polymer carrier such as disclosed in U.S. Pat. No. 5,633,001, entitled “Composition and a Method for Tissue Augmentation”.

Filler compositions as disclosed supra or combinations thereof can further include compositions with materials that aid in growth or suppress growth of the injected or surrounding tissues. For example such embodiments can include compositions comprising autologous body components and fluids as disclosed in co-owned U.S. patent Ser. No. 11/210,273, entitled “Methods of Administering Microparticles Combined With Autologous Body Components”. Alternatively, one can prepare a composition comprising cells and a filler material. Cells can be autogeneic, isogeneic, allogeneic or xenogeneic. Cells can be genetically engineered. The compositions can contain different cell types, which can be chosen to act synergistically, for example, in the formation of tissue. Examples of types of cells include muscle cells, nerve cells, epithelial cells, connective tissue cells, and organ cells. Specific examples of cells include fibroblast cells, smooth muscle cells, striated muscle cells, heart muscle cells, nerve cells, epithelial cells, endothelial cells, bone cells, bone progenitor cells, bone marrow cells, blood cells, brain cells, kidney cells, liver cells, lung cells, pancreatic cells, spleen cells, breast cells, foreskin cells, ovary cells, testes cells and prostate cells. The types of cells include stem cells, which can be fetal stem cells or adult stem cells and can be totipotent, multipotent, or pluripotent. Other mammalian cells are useful in the practice of the invention and are not excluded from consideration here. Alternatively, the filler material compositions can include non-mammalian eukaryotic cells, prokaryotic cells or viruses.

Filler compositions can include physiologically buffered salt solutions, water, glycerol and the like, and can be supplemented with, for example, serum, growth factors, hormones, sugars, amino acids, vitamins, metalloproteins, lipoproteins, and the like.

Growth factors include, but are not limited to, transforming growth factors (TGFs), fibroblast growth factors (FGFs), platelet derived growth factors (PDGFs), epidermal growth factors (EGFs), connective tissue activated peptides (CTAPs), osteogenic factors, and biologically active analogs, fragments, and derivatives of such growth factors. Members of the TGF supergene family include the beta transforming growth factors (for example, TGF-.beta.1, TGF-.beta.2, TGF-.beta.3); bone morphogenetic proteins (for example, BMP-1, BMP-2, BMP-3, BMP-4, BMP-5, BMP-6, BMP-7, BMP-8, BMP-9); heparin-binding growth factors (for example, fibroblast growth factor (FGF), epidermal growth factor (EGF), platelet-derived growth factor (PDGF), insulin-like growth factor (IGF)); Inhibins (for example, Inhibin A, Inhibin B); growth differentiating factors (for example, GDF-1); and Activins (for example, Activin A, Activin B, Activin AB).

Growth factors can be isolated from native or natural sources, such as from mammalian cells, or can be prepared synthetically, such as by recombinant DNA techniques or by various chemical processes. In addition, analogs, fragments, or derivatives of these factors can be used, provided that they exhibit at least some of the biological activity of the native molecule. For example, analogs can be prepared by expression of genes altered by site-specific mutagenesis or other genetic engineering techniques.

A solution, mixture, or suspension comprising a filler, for example, ArteFill® or other collagen-based or other injectable material, which is suitable, for example, for use in combination with any of the preceding operative procedures, can in representative applications be stored in a syringe or other container (e.g., supplier-provided container or storage container) for relatively long (e.g., extended) periods of time before the operative procedure is performed.

A filler, such as ArteFill® or other collagen-based composition or other injectable material, wherein the syringe or container is made of or comprises a barrier material that is substantially impermeable to the solution or mixture so that the solution or mixture does not leak, or does not substantially leak, through the walls of the syringe or other container when the solution or mixture (e.g., injectable) is stored in the syringe or other container for relatively extended periods of time. The impermeability of the syringe or container can approach, be comparable, match, or exceed that of a similarly sized and/or shaped syringe or container formed from glass.

According to another aspect of the present invention, a syringe or other container for storing solutions or mixtures (e.g., suspensions) comprising, for example, ArteFill® or other collagen-based or other injectables, in any combination, is provided, wherein the syringe or container can be formed from, or can comprise, a barrier material that does not contain substantially leachable heavy metals, and/or does not leach or substantially leach heavy metals into the solution or mixture stored in the syringe or other container. The attenuation of or resistance to leaching of heavy metals of the syringe or container can exceed that of a similarly sized and/or shaped syringe or container formed from glass.

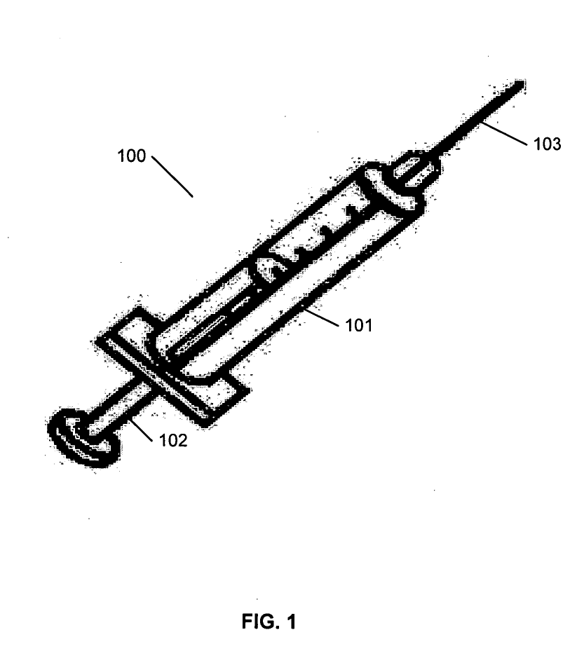

In certain embodiments the syringe 100 can comprise the components of a barrel 101, a plunger 102 and a needle 103 as shown in FIG. 1. One or more of the component parts can be formed from, or can comprise, a barrier material that does not contain substantially leachable heavy metals, and/or does not leach or substantially leach heavy metals into the solution or mixture stored in the syringe or other container. In certain embodiments, one or more components comprise liquid crystal polymer.

In certain embodiments, the syringe can have an attached lumen. An implementation of such a syringe and lumen can comprise, for example, the injection facilitation apparatus described in U.S. Pat. No. 6,666,848, incorporated by reference herein. Said syringe and lumen can be provided in a modified form to comprise, for example, a barrier material that is both relatively impermeable to the injectable stored or contained therein over extended time periods and that does not contain substantially leachable heavy metals which may leach into the injectable stored therein over extended time periods.

In another embodiment of the invention, a syringe can comprise multiple stoppers, such as that disclosed in the co-owned U.S. patent Ser. No. 11/325,618, entitled “Syringe with a Plurality of Stoppers” and incorporated by reference herein, and can be provided in a modified form to include, for example, a barrier material that is both relatively impermeable to the injectable stored or contained therein over extended time periods and that does not contain substantially leachable heavy metals which may leach into the injectable stored therein over extended time periods.

In accordance with an embodiment of the present invention, a syringe or other container comprises a barrier material, which can be at least one liquid crystal polymer (LCP). LCP compositions provide various advantageous properties including, but not limited to, barriers against moisture and vapor transmission, high optical clarity, high scratch resistance, and inertness. In particular instances of the present invention, one or more of the liquid crystal polymers are selected from those categorized as United States Pharmacopeia (USP) Class VI materials, which have been tested in animal studies and been approved by the Food and Drug Administration (FDA) for use in long-term animal implants. Exemplary embodiments can include a syringe or another container comprising at least one liquid crystal polymer selected from the group consisting of ZYLAR®, ZENITE® and TOPAS®. ZYLAR® is the registered trademark for styrene methylmethacrylate co polymer, commercially available from Nova Chemicals of Calgary, Canada, or from General Polymers of Cincinnati, Ohio. Zenite® is the registered trademark for a proprietary formulation of wholly aromatic polyester resins commercially available from DuPont Engineering Polymers of Wilmington, Del. TOPAS® is the registered trademark for the cyclic olefin copolymer (COP) of ethylene and norbornene. TOPAS® is commercially available from Ticona Engineering Polymers of Florence, Ky. Ticona Engineering Polymers is a division of Celanese Corporation.

The barrier materials (e.g., liquid crystal polymers) can comprise thermoplastics or thermosets. In particular implementations, liquid crystal polymers may be essentially impermeable to materials (e.g., liquid materials) such as materials constituting or forming parts of the referenced solutions or mixtures over extended time periods. Accordingly, materials (e.g., liquid materials) forming or combined in any way with the solutions or mixtures, comprising, for example, ArteFill® or other collagen-based or other injectable solutions or mixtures, can be stored in syringes or other containers that comprise at least one barrier material, such as one or more liquid crystal polymers, whereby minimal leaching of the stored materials through the walls of the syringe or other container can be attenuated or eliminated. Said injectable compositions can be stably stored in pre-filled syringes or containers for a week, a month, or a year. Based on stability data, stable storage can potentially be obtained for two years, five years, ten years or twenty years or more.

The barrier material, such as a liquid crystal polymer composition, can be layered onto the interior portion of one or more components of a syringe or container. Example methods and compositions for layering barrier materials are disclosed in U.S. Pat. No. 5,939,153, entitled “Multilayered Plastic Container” herein incorporated by reference. The barrier materials can also comprise a rigid sleeve to be inserted into the syringe or container. Said sleeve can be deformable, comprising compositions such as disclosed in U.S. Pat. No. 6,284,333, entitled “Medical Devices Made From Polymer Blends Containing Low Melting Temperature Liquid Crystal Polymers” herein incorporated by reference. Said deformable sleeve can be enclosed to prevent contact of sleeve contents with syringe components such as the barrel and plunger, but allow injection of sleeve contents. The addition of such a deformable sleeve to a syringe allows for the long term, stable storage of injectable compositions.

In other aspects of the present invention, syringes or other containers comprise, consist essentially of, or consist of, barrier materials (e.g., non-glass barrier material or barrier materials, such as polymer materials or in exemplary instances liquid crystal polymers including, for instance, USP Class VI liquid crystal polymers) that are functionally equivalent or substantially equivalent, in whole or in part, to glass for preventing leaching of solutions or materials out of the syringes or containers during storage of the solutions or materials within the syringes or containers for relatively extended periods of time. The barrier materials can be functionally equivalent or substantially equivalent to glass for preventing leaching of solutions or materials out of the syringes or containers during storage of the solutions or materials within the syringes or containers for relatively extended periods of time. Furthermore, the barrier materials may leach heavy metals to a lesser extent (e.g., a negligible or non-measurable extent) than glass.

In further aspects of the present invention, the syringes or other containers comprise, consist essentially of, or consist of, barrier materials (e.g., non-glass barrier material or barrier materials, such as polymer materials or in certain implementations liquid crystal polymers including, for instance, USP Class VI liquid crystal polymers) having vapor or moisture barriers, or one or more vapor or moisture barrier characteristics, that are functionally about the same as (e.g., substantially equivalent to or equivalent to), in whole or in part, glass for preventing leaching of solutions or materials out of the syringes or containers during storage of the solutions or materials within the syringes or containers for relatively extended periods of time. The barrier materials can have vapor and moisture barriers, or one or more vapor or moisture barrier characteristics, that are functionally about the same as glass for preventing leaching of solutions or materials out of the syringes or containers during storage thereof within the syringes or containers for relatively extended periods of time.

Table 1 shows water vapor permeability of standard syringe barrels and syringe barrels made from early formulations of liquid crystal polymers. Table 2 shows water vapor permeability of newer liquid crystal polymers. Data from Plastics Engineering, by R J CRAWFORD, Butterworth-Heinemann; 3d edition (Mar. 9, 1998), p. 36. Liquid crystal polymers have a much reduced water vapor permeability as compared to other commonly used materials for syringe barrels.

TABLE 1
Water Vapor Permeabilities of Typical Syringe Barrel Materials vs. Liquid
Crystal Polymers

| | |
|---|---|
| Polypropylenes | ˜10 (grams 25 μm/m²-24 hours-atm) |
| High Density Polyethylenes | ˜10 (grams 25 μm/m²-24 hours-atm) |
| Polycarbonates | ˜120 (grams 25 μm/m²-24 hours-atm) |
| Liquid Crystal Polymers (early | ˜0.1 (grams 25 μm/m²-24 hours-atm) |
| formulations) | |

TABLE 2
Water Vapor Permeabilities of New Generation Liquid Crystal Polymers

| | |
|---|---|
| Zylar (NOVA Chemicals) | 0.1 (grams mm/m²-Day-atm @85% |
| | RH) |
| Topas (Celanese Corporation) | 0.023 (grams mm/m²-Day-atm @85% |
| | RH) |

In another aspect of the invention, methods for storing filler material by filling a syringe or container made of, at least in part, a liquid crystal polymer with filler material and sealing the syringe to prevent escape of filler material. The sealing step can include using syringe components that have liquid crystal polymer compositions coating the filler material contacting surfaces, such as a barrel, an interior sleeve, a plunger, or a needle, individually or in combination. Alternatively or in combination, other sealing parts can include caps, covers, tape, and the like. The filler can be polymethylmethacrylate (PMMA) microspheres. The PMMA microspheres can be formulated with collagen. Such formulations can be ArteFill®.

### EXAMPLE 1

### Long-Term, Stable Storage Study of ArteFill® in 1 cc Zylar Syringes Versus Becton Dickinson 1 cc Polypropylene Syringes

The long-term, stable storage of ArteFill® in LCP syringes was investigated. 20 each of Zylar syringes from Merit Medical (South Jordan, Utah) and 20 each of Polypropylene syringes from Becton, Dickinson and Company (Franklin Lakes, N.J.) were filled with an average of 0.85 cc of ArteFill® (Artes Medical Inc., San Diego, Calif.) and placed in a refrigerator at 4 degrees C. The level of ArteFill® remaining in each syringe was visually monitored and also determined by weight using a Mettler Toledo Analytical Balance (Columbus, Ohio) with 0.0001 gram resolution. After 12 months of time there was an average loss of 22.8% of the original mass of ArteFill® from the Becton Dickinson polypropylene syringes, and an average loss of 4.6% of the original mass of ArteFill® was lost from the Merit Medical Zylar syringes. It was also observed that the BD syringes exhibited voids all along the barrel walls, while the Zylar syringes did not. Both syringe types exhibited voids beneath the caps, indicating that the bulk of the moisture loss from the Zylar syringes was occurring through the syringe caps, not through the barrel walls.

The present invention has been described above in terms of various embodiments so that an understanding of the present invention can be conveyed. There are, however, many embodiments for syringes and containers not specifically described herein but with which the present invention is applicable. The present invention should therefore not be seen as limited to the particular embodiments described herein, but rather, those skilled in the art will appreciate that additional materials, techniques, or combinations of materials and techniques can be used to achieve the advantages of the invention. The invention is identified by the following claims.

I claim:

1. A pre-filled syringe comprising a barrel and a filler composition, wherein said barrel comprises liquid crystal polymer.

2. The pre-filled syringe of claim 1, wherein said liquid crystal polymer comprises one or more of styrene methylmethacrylate co polymer, Zenite®, or cyclic olefin copolymer (COP) of ethylene and norbornene.

3. The pre-filled syringe of claim 2, wherein said barrel consists essentially of liquid crystal polymer.

4. The pre-filled syringe of claim 1, wherein said filler composition is a soft tissue filler.

5. The pre-filled syringe of claim 4, wherein said soft tissue filler comprises PMMA microspheres.

6. The pre-filled syringe of claim 5, wherein said soft tissue filler comprises collagen.

7. The pre-filled syringe of claim 5, wherein said soft tissue filler comprises approximately 20% by weight PMMA and approximately 80% by weight a composition comprising 3.5% purified bovine collagen, 2.7% phosphate buffer, 0.9% sodium chloride, 0.3% lidocaine hydrochloride, and 92.6% water for injection.

8. The pre-filled syringe of claim 1, further comprising a plunger, wherein contents-contacting surfaces of said plunger comprise a liquid crystal polymer.

9. The pre-filled syringe of claim 8, wherein said liquid crystal polymer comprises one or more of styrene methylmethacrylate co polymer, ZENITE®, or cyclic olefin copolymer (COP) of ethylene and norbornene.

10. The pre-filled syringe of claim 8, wherein said plunger consists essentially of liquid crystal polymer.

11. The pre-filled syringe of claim 8, wherein said filler composition is a soft tissue filler.

12. The pre-filled syringe of claim 11, wherein said soft tissue filler comprises PMMA microspheres.

13. The pre-filled syringe of claim 11, wherein said soft tissue filler comprises collagen.

14. The pre-filled syringe of claim 12, wherein said soft tissue filler comprises approximately 20% by weight PMMA and approximately 80% by weight a composition of 3.5% purified bovine collagen, 2.7% phosphate buffer, 0.9% sodium chloride, 0.3% lidocaine hydrochloride, and 92.6% water for injection.

15. The pre-filled syringe of claim 1, further comprising a needle, wherein contents-contacting surfaces of said needle comprises a liquid crystal polymer.

16. The pre-filled syringe of claim 15, wherein said liquid crystal polymer comprises one or more of styrene methylmethacrylate co polymer, ZENITE®, or cyclic olefin copolymer (COP) of ethylene and norbornene.

17. The pre-filled syringe of claim 15, wherein said needle consists essentially of liquid crystal polymer.

18. The pre-filled syringe of claim 15, wherein said filler composition is a soft tissue filler.

19. The pre-filled syringe of claim 18, wherein said soft tissue filler comprises PMMA microspheres.

20. The pre-filled syringe of claim 18, wherein said soft tissue filler comprises collagen.

21. The pre-filled syringe of claim 19, wherein said soft tissue filler comprises approximately 20% by weight PMMA and 80% by weight a composition of 3.5% purified bovine collagen, 2.7% phosphate buffer, 0.9% sodium chloride, 0.3% lidocaine hydrochloride, and 92.6% water for injection.

22. A pre-filled syringe comprising a barrel, an interior sleeve, and a filler composition, wherein said sleeve comprises liquid crystal polymer.

23. The pre-filled syringe of claim 22, wherein said liquid crystal polymer comprises one or more of styrene methylmethacrylate co polymer, ZENITE®, or cyclic olefin copolymer (COP) of ethylene and norbornene.

24. The pre-filled syringe of claim 22, wherein said interior sleeve consists essentially of liquid crystal polymer.

25. The pre-filled syringe of claim 22, wherein said filler composition is a soft tissue filler.

26. The pre-filled syringe of claim 25, wherein said soft tissue filler comprises PMMA microspheres.

27. The pre-filled syringe of claim 25, wherein said soft tissue filler comprises collagen.

28. The pre-filled syringe of claim 26, wherein said soft tissue filler comprises approximately 20% by weight PMMA and 80% by weight a composition of 3.5% purified bovine collagen, 2.7% phosphate buffer, 0.9% sodium chloride, 0.3% lidocaine hydrochloride, and 92.6% water for injection.

29. The pre-filled syringe of claim 24, wherein said interior sleeve is deformable.

30. A method for the stable storage of a filler material comprising filling a syringe comprising a liquid crystal polymer with filler material and sealing said syringe.

31. The method of claim 30, wherein said liquid crystal polymer comprises one or more of styrene methylmethacrylate co polymer, ZENITE®, or cyclic olefin copolymer (COP) of ethylene and norbornene.

32. The method of claim 30, wherein said filler composition is a soft tissue filler.

33. The method of claim 30, wherein said soft tissue filler comprises PMMA microspheres.

34. The method of claim 30, wherein said soft tissue filler comprises collagen.

35. The method of claim 33, wherein said soft tissue filler comprises approximately 20% by weight PMMA and approximately 80% by weight a composition of 3.5% purified bovine collagen, 2.7% phosphate buffer, 0.9% sodium chloride, 0.3% lidocaine hydrochloride, and 92.6% water for injection.
